(19) United States
(12) Patent Application Publication
(10) Pub. No.: US 2026/0183557 A1
(43) Pub. Date: Jul. 2, 2026

(54) Electrode Assemblies for Applying Tumor Treating Fields (TTFields) to a Subject's Body with a Strain Relief That Absorbs Cable-Induced Strain

(71) Applicant: Novocure GmbH, Baar (CH)

(72) Inventors: Elie YAACOBI, Haifa (IL); Noa HALAVEE, Haifa (IL); Dmitry GOLOM, Haifa (IL)

(73) Assignee: Novocure GmbH, Baar (CH)

(21) Appl. No.: 19/436,010

(22) Filed: Dec. 30, 2025

Related U.S. Application Data
(60) Provisional application No. 63/740,917, filed on Dec. 31, 2024.

(51) Int. Cl. A61N 1/40; A61N 1/04

(52) U.S. Cl. CPC A61N 1/40; A61N 1/0492

(57) ABSTRACT

Tumor Treating Fields (TTFields) therapy is a proven approach for treating tumors using alternating electric fields. The TTFields are induced by electrode assemblies positioned on the subject's skin. When the electrode assemblies are implemented using flex circuits, the place where the cable terminates on the flex circuit is susceptible to mechanical failure. By incorporating a flex circuit that is bent back on itself so that one region of the PCB is positioned behind another region of the PCB, the embodiments described herein absorb mechanical strain that is induced by pulling upon the cables before those strains can reach the flex circuit. And this advantageously reduces the incidence of mechanical failures.

### CROSS REFERENCE TO RELATED APPLICATIONS

This Application claims the benefit of U.S. Provisional Application 63/740,917 filed Dec. 31, 2024, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 kHz. The alternating electric fields are induced by electrode assemblies (also called transducer arrays) positioned on the subject's skin on opposite sides of the subject's body, and an electrical signal is delivered to each of the electrode assemblies by a respective cable. When an AC voltage is applied between opposing electrode assemblies (via these cables), an AC current is coupled through the electrode assemblies and into the subject's body, which induces the TTFields in a target region within the subject's body.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

U.S. Pat. No. 12,114,991 describes some different prior art electrode assemblies that can be used to apply alternating electric fields to a subject's body. In one example, each electrode assembly includes a flex circuit that includes a plurality of conductive pads on the front side of the flex circuit, and these conductive pads serve as electrode elements.

### SUMMARY OF THE INVENTION

One aspect of the invention is directed to a first apparatus for applying an electrical signal to a subject's body. The first apparatus comprises a flexible backing, a flexible PCB, a sheet of graphite, and a cable. The flexible backing has a front surface. The flexible PCB is affixed to the front surface of the flexible backing by a first layer of adhesive. The PCB has (i) a first region having at least one metal pad disposed on one surface of the PCB, (ii) a second region that is contiguous with the first region and extends from the first region in a first direction, and (iii) a third region that is contiguous with the second region and extends from the second region in a second direction. The PCB is oriented so that the at least one metal pad is front-facing. And the PCB is bent back on itself about a bending axis that passes through the second region so that the third region is positioned behind the first region. The sheet of graphite is positioned in front of the PCB, and the sheet of graphite is affixed to the PCB by a layer of conductive adhesive. The cable terminates on the third region of the PCB, and the cable is affixed to the flexible backing and/or the sheet of graphite and/or the PCB by at least one layer of adhesive.

In some embodiments of the first apparatus, the at least one layer of adhesive comprises the first layer of adhesive. In some embodiments of the first apparatus, the at least one layer of adhesive comprises the layer of conductive adhesive. In some embodiments of the first apparatus, the at least one layer of adhesive comprises both the first layer of adhesive and the layer of conductive adhesive. In some embodiments of the first apparatus, the cable is affixed to the sheet of graphite and/or the PCB by double-sided foam tape that has a front layer of adhesive and a rear layer of adhesive, and at least a portion of the double-sided foam tape is situated between the first region of the PCB and the third region of the PCB.

In some embodiments of the first apparatus, the sheet of graphite has at least one slit disposed therein, and the at least one slit is positioned to increase flexibility of the first apparatus when the first apparatus is adhered to the subject's body. Optionally, these embodiments can further comprise a layer of flexible foam material shaped, dimensioned, and positioned to cover a front side of all edges of the sheet of graphite and the at least one slit.

In some embodiments of the first apparatus, the sheet of graphite has at least one narrow cutout disposed therein, and the at least one narrow cutout is positioned to increase flexibility of the first apparatus when the first apparatus is adhered to the subject's body.

In some embodiments of the first apparatus, the PCB is bent back on itself with a bending radius that is at least 6 times a thickness of the PCB. In some embodiments of the first apparatus, the bending axis is substantially perpendicular to the first direction.

In some embodiments of the first apparatus, the bending axis is substantially perpendicular to the first direction and the second direction is substantially perpendicular to the first direction. Optionally, in these embodiments, the cable can extend in the second direction from the third region of the PCB.

In some embodiments of the first apparatus, the bending axis is substantially perpendicular to the first direction, and the second direction is substantially parallel to the first direction. In some embodiments of the first apparatus, the sheet of graphite has an area of 50-150 cm².

Another aspect of the invention is directed to a second apparatus for applying an electrical signal to a subject's body. The second apparatus comprises a flexible backing, a flexible PCB, a sheet of graphite, and a connector. The flexible backing has a front surface.

The flexible PCB is affixed to the front surface of the flexible backing by a first layer of adhesive. The PCB has (i) a first region having at least one metal pad disposed on one surface of the PCB, (ii) a second region that is contiguous with the first region and extends from the first region in a first direction, and (iii) a third region that is contiguous with the second region and extends from the second region in a second direction. The PCB is oriented so that the at least one metal pad is front-facing. And the PCB is bent back on itself about a bending axis that passes through the second region so that the third region is positioned behind the first region. The sheet of graphite is positioned in front of the PCB, and the sheet of graphite is affixed to the PCB by a layer of conductive adhesive. The connector terminates on the third region of the PCB, and the connector is affixed to the flexible backing and/or the sheet of graphite and/or the PCB by at least one layer of adhesive.

In some embodiments of the second apparatus, the at least one layer of adhesive comprises the first layer of adhesive. In some embodiments of the second apparatus, the at least one layer of adhesive comprises the layer of conductive adhesive. In some embodiments of the second apparatus, the at least one layer of adhesive comprises both the first layer of adhesive and the layer of conductive adhesive. In some embodiments of the second apparatus, the connector is affixed to the sheet of graphite and/or the PCB by double-sided foam tape that has a front layer of adhesive and a rear layer of adhesive, and at least a portion of the double-sided foam tape is situated between the first region of the PCB and the third region of the PCB.

In some embodiments of the second apparatus, the sheet of graphite has at least one slit disposed therein, and the at least one slit is positioned to increase flexibility of the second apparatus when the second apparatus is adhered to the subject's body. Optionally, these embodiments can further comprise a layer of flexible foam material shaped, dimensioned, and positioned to cover a front side of all edges of the sheet of graphite and the at least one slit.

In some embodiments of the second apparatus, the sheet of graphite has at least one narrow cutout disposed therein, and the at least one narrow cutout is positioned to increase flexibility of the second apparatus when the second apparatus is adhered to the subject's body.

In some embodiments of the second apparatus, the PCB is bent back on itself with a bending radius that is at least 6 times a thickness of the PCB. In some embodiments of the second apparatus, the bending axis is substantially perpendicular to the first direction.

In some embodiments of the second apparatus, the bending axis is substantially perpendicular to the first direction and the second direction is substantially perpendicular to the first direction. Optionally, in these embodiments, the connector extends in the second direction from the third region of the PCB.

In some embodiments of the second apparatus, the bending axis is substantially perpendicular to the first direction and the second direction is substantially parallel to the first direction. In some embodiments of the second apparatus, the sheet of graphite has an area of 50-150 cm².

### BRIEF DESCRIPTION OF THE DRAWINGS

FIG. 1 depicts an exploded view of an electrode assembly that, when positioned on a subject's body, can be used to induce TTFields in the subject's body.

FIG. 2A depicts a rear view of the FIG. 1 embodiment with the flexible backing removed so that the components in front of the flexible backing can be seen.

FIG. 2B depicts a front view of the FIG. 1 embodiment with the cover removed so that the components positioned behind the cover can be seen.

FIG. 3 is a more detailed view of the FIG. 1 embodiment with the flexible backing removed.

FIGS. 4A and 4B are detail views of a portion of the FIG. 1 embodiment at two different stages of the manufacturing process.

FIGS. 5A and 5B depict another electrode assembly that, when positioned on a subject's body, can be used to induce TTFields in the subject's body.

Various embodiments are described in detail below with reference to the accompanying drawings, wherein like reference numerals represent like elements.

### DESCRIPTION OF THE PREFERRED EMBODIMENTS

The inventors recognized that when the electrode assemblies are implemented using flex circuits, the place where the cable terminates on the flex circuit is the part of the system that is most prone to mechanical failure. The embodiments described below absorb mechanical strain that is induced by pulling upon the cables before those strains can reach the flex circuit. And this advantageously reduces the incidence of mechanical failures.

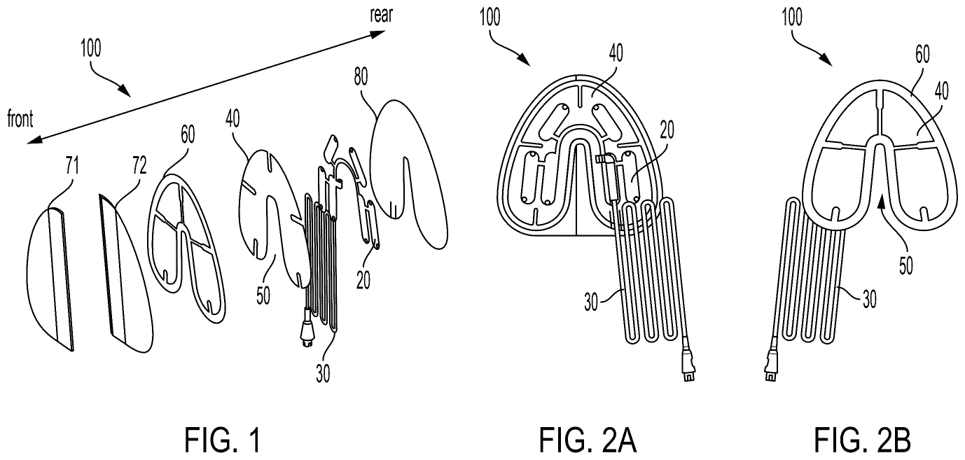

FIG. 1 depicts an exploded view of an electrode assembly 100 that, when positioned on a subject's body, can be used to induce TTFields in the subject's body. The electrode assembly 100 includes (progressing from the rear to the front) a flexible backing 80, a flexible PCB 20 (i.e., a flexible printed circuit board, which is commonly referred to as a “flex circuit”) with a cable 30 that terminates on the flexible PCB 20, a sheet of graphite 40 (shown with a gap 50), an optional layer of foam material 60, and a cover 71, 72 (such as, for example, a release liner). As used herein, the front of the electrode assembly 100 faces the subject's body during use, and the rear of the electrode assembly 100 faces away from the subject's body. These directions are labeled near the top of FIG. 1. During use, the cover 71, 72 is removed.

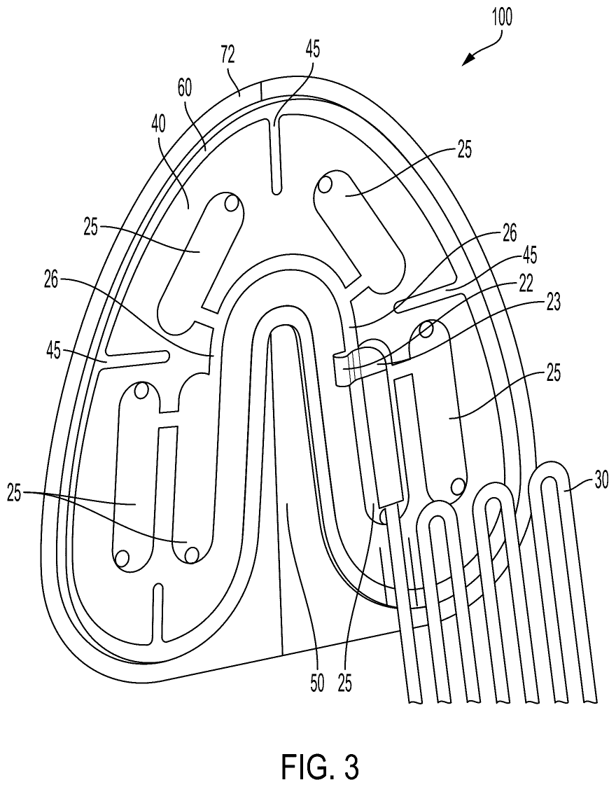

FIG. 2A depicts a rear view of the electrode assembly 100 with the flexible backing 80 removed so that the components in front of the flexible backing 80 can be seen. FIG. 2B depicts a front view of the electrode assembly 100 with the cover 71, 72 removed so that the components positioned behind the cover 71, 72 can be seen. And FIG. 3 is a more detailed view of the electrode assembly 100 with the flexible backing 80 removed.

Referring now to FIG. 1, the flexible backing 80 has a front surface and can be made of a variety of materials including but not limited to flexible fabric materials, foam materials, and plastic materials (e.g., similar to corresponding varieties of Band-Aid® brand adhesive bandages). A flexible PCB 20 is affixed to the front surface of the flexible backing 80 by a first layer of adhesive. This first layer of adhesive could be, for example, a self-adhesive front surface of the flexible backing 80.

As best seen in FIG. 3, the PCB 20 has (i) a large first region having at least one metal pad 25 disposed on one surface of the PCB, (ii) a small second region 22 that is contiguous with the first region and extends from the first region in a first direction, and (iii) a third region 23 that is contiguous with the second region 22 and extends from the second region in a second direction. The first region of the PCB 20 is the region that includes all of the metal pads 25 and the conductive traces 26 that run between those metal pads, as well as the portion of the flexible insulating substrate that supports those pads 25 and traces 26. The PCB 20 is oriented so that the at least one metal pad 25 is front-facing. The metal pads 25 can be, e.g., copper pads that may optionally have an ENIG or ENEPIG coating (i.e., Electroless Nickel Immersion Gold or Electroless Nickel Electroless Palladium Immersion Gold coating). Although the first region of the PCB 20 is illustrated with six metal pads, the number and shape of the metal pads may vary, and, separately, the footprint of the conductive traces 26 may also vary.

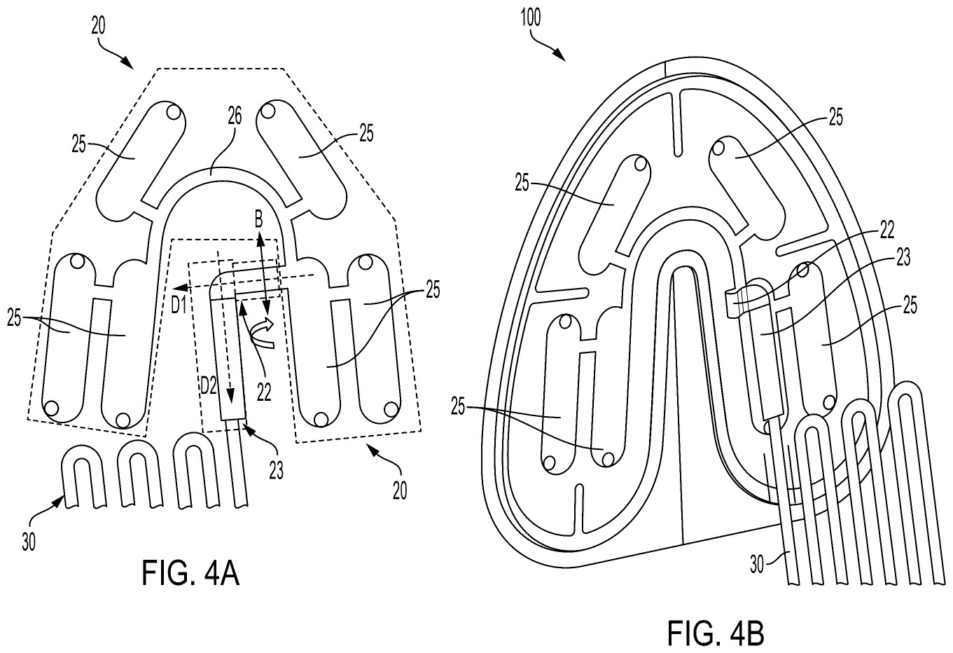

FIG. 4A is a detailed view of the first, second, and third regions of the PCB at an early stage of the manufacturing process, prior to the point where the PCB 20 has been bent into the configuration described above and depicted in FIG. 3. The first region of the PCB 20 includes the at least one metal pad 25 and the conductive traces 26 (facing away from the reader in FIG. 4A). The second region 22 is contiguous with the first region and extends from the first region in a first direction D1. And the third region 23 is contiguous with the second region 22 and extends from the second region in a second direction D2. During the manufacturing process, the PCB 20 is bent back on itself about a bending axis (labeled B) that passes through the second region 22 so that the third region 23 moves toward the reader in FIG. 4A, until the third region 23 reaches the position depicted in FIG. 4B. At this point, the third region 23 will be positioned behind the first region of the PCB 20 (i.e., closer to the reader because FIG. 4A is a rear view of the device). In some preferred embodiments, the bending radius of the bend that runs through the second region 22 is at least 6 times a thickness of the PCB.

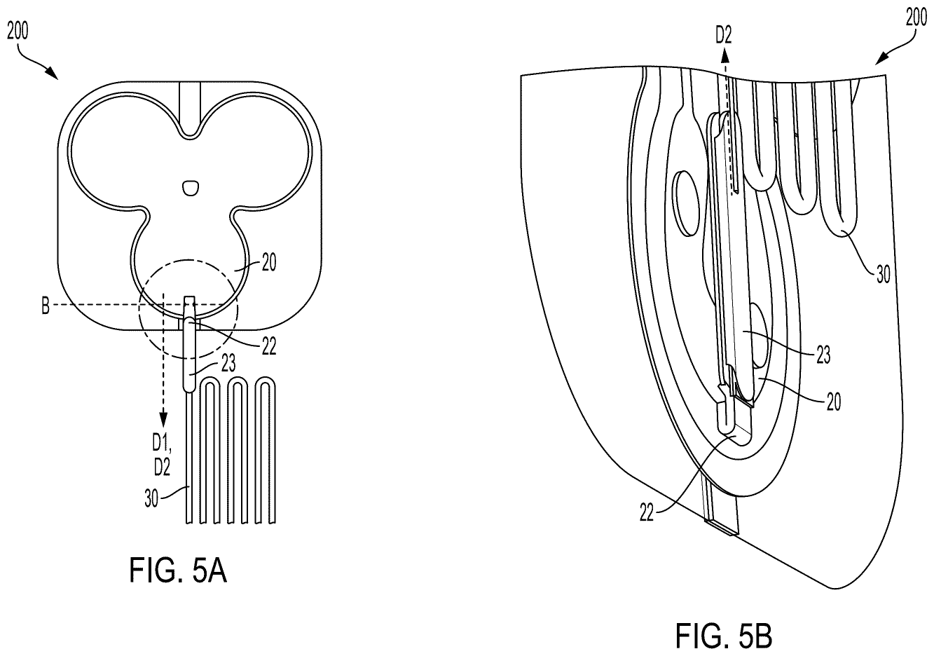

In the example illustrated in FIG. 4A, the bending axis B is substantially perpendicular to the first direction D1, and the second direction D2 is substantially perpendicular to the first direction. But this is not the only possible configuration. For example, in the FIGS. 1-4 embodiment, the depicted configuration could be replaced with a configuration in which the bending axis B is substantially perpendicular to the first direction D1 and the second direction D2 is substantially parallel to the first direction D1. Such a configuration would be similar to the configuration described below in connection with FIGS. 5A-B.

At a subsequent time in the manufacturing process, the cable 30 is connected (e.g., by soldering) so that it terminates on the third region 23 of the PCB 20, as depicted in FIG. 4B. Note that in the example depicted in FIG. 4B, the cable 30 extends in the second direction D2 from the third region 23 of the PCB 20. Conductive wires within the cable 30 and traces (including but not limited to the traces 26) on the PCB 20 form the paths over which the signals from an AC signal generator (not shown) arrive at the metal pads 25 in order to apply TTFields to the region of interest in the subject's body.

Returning now to FIG. 1, a sheet of graphite 40 is positioned in front of the PCB 20, and the sheet of graphite 40 is affixed to the PCB by a layer of conductive adhesive.

Examples of suitable materials for the sheet of graphite 40 include, but are not limited to, synthetic graphite, pyrolytic graphite (including, but not limited to, Pyrolytic Graphite Sheet (PGS), available from Panasonic Industry, Kadoma, Osaka, Japan), graphitized polymer film (e.g., graphitized polyimide film, including, but not limited to, that supplied by Kaneka Corp., Moka, Tochigi, Japan), or graphite foil made from compressed high purity exfoliated mineral graphite (including, but not limited to, that supplied by MinGraph® 2010A Flexible Graphite, available from Mineral Seal Corp., Tucson, Arizona, USA). The sheet of graphite 40 spreads out both heat and current in the plane of the page in FIGS. 2A and 2B. In some embodiments, a layer of a different conductive anisotropic material may be used in place of the sheet of graphite 40.

The sheet of graphite 40 in this FIG. 3 embodiment is generally shaped like a rounded V. But a wide variety of alternative shapes for the sheet of graphite 40 may be used, including but not limited to rounded U shapes, C shapes, rectangular shapes, rounded rectangular shapes, clover-leaf shapes, etc. In some embodiments, the sheet of graphite 40 has an area between 50 and 150 cm² or between 75 and 125 cm². But in other embodiments, the sheet of graphite 40 can have an area of 75-100 cm², 75-150 cm², 75-200 cm², 75-250 cm², 75-300 cm², 50-100 cm², 50-125 cm², 50-200 cm², 50-250 cm², 50-300 cm², 30-100 cm², 30-125 cm², 30-150 cm², 30-200 cm², or 30-300 cm², or even an area that is not within any of these ranges.

The sheet of graphite 40 depicted in FIG. 3 has five slits 45 disposed therein (one is obscured by the cable 30), and these slits are positioned to increase flexibility of the electrode assembly 100 when the electrode assembly is adhered to a subject's body. Note, however, that a wide variety of different configurations for the slits can be used in place of the configuration of slits 45 depicted in FIG. 3. In alternative embodiments (not shown), a narrow cutout that is significantly wider than the illustrated slits 45 can be used instead of the slits 45 depicted in FIG. 3. Other configurations for the slits 45 may utilize a different number of slits. For example, instead of five slits 45, in some embodiments, the sheet of graphite 40 may have three slits 45, which may or may not occupy three of the five locations shown for the five slits in FIG. 3. By way of example, and without limitation, in FIG. 3, one could omit the two slits 45 on the distal ends of the two extended arms; or, alternatively, one could omit the two slits 45 on the outer perimeter of the two arms toward the apex of the gap 50.

Examples of suitable materials for the layer of conductive adhesive that affixes the sheet of graphite 40 to the PCB 20 include, but are not limited to, the OMNI-WAVE™ adhesive compositions manufactured and sold by FLEXCON® (Spencer, MA, USA), such as the developmental product FLX068983-FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M, Saint Paul, MN, USA) may also be used. In other embodiments, the sheet of graphite 40 may be affixed to the PCB 20 by a layer of conductive hydrogel.

Note that the sheet of graphite 40 can be affixed to the PCB 20 by positioning only a single layer of conductive adhesive between the sheet of graphite 40 and the PCB 20 as described above. But in alternative embodiments, additional layers of conductive material can be positioned between the sheet of graphite 40 and the PCB 20. For example, two layers made from different types of conductive adhesives (i.e., a front layer and a rear layer) can be positioned between the sheet of graphite 40 and the PCB 20. In this situation, the front layer affixes the sheet of graphite 40 to the PCB 20, and the rear layer also affixes the sheet of graphite 40 to the PCB 20. Alternatively, a layer of conductive adhesive (front layer or rear layer) and a layer of conductive hydrogel (front layer or rear layer) may be used.

Optionally, and as best seen in FIGS. 1 and 2B, the electrode assembly 100 also has a layer of flexible foam material 60 shaped, dimensioned, and positioned to cover a front side of all edges of the sheet of graphite 40, and all of the slits 45. The purpose of this layer of flexible foam material 60 is to cover all the edges of the sheet of graphite 40 because those edges can be sharp, and covering those edges can prevent the subject from getting small cuts. This foam material can be similar to the foam material that is used in foam self-adhesive bandages.

Preferably, a front layer of conductive adhesive or conductive hydrogel (not shown) is disposed on the front face of the sheet of graphite 40, and this layer will help the electrode assembly 100 adhere to the subject's skin. The same materials described above in connection with the conductive adhesive that affixes the sheet of graphite 40 to the PCB 20 can be used on the front face of the sheet of graphite. In some embodiments (not shown), the flexible foam material 60 is omitted, and the front layer of conductive adhesive described in this paragraph is dimensioned to be slightly larger than the sheet of graphite 40 in all directions. This configuration can help prevent the subject from getting the small cuts that are discussed in the previous paragraph.

The electrode assembly 100 depicted in FIGS. 1-3 also has a front cover 71, 72 positioned in front of the layer of conductive adhesive (or hydrogel) that sits in front of the sheet of graphite 40. This cover 71, 72 (e.g., release liner) performs a similar function to the coated-paper slips that cover Band-Aid® brand adhesive bandages, and it prevents dust and dirt from settling on the front layer of adhesive before the electrode assembly 100 is applied to the subject's skin. Note, however, that in alternative embodiments, the cover 71, 72 can be omitted.

As explained above in connection with FIG. 4B, the cable 30 is connected (e.g., by soldering) so that it terminates on the third region 23 of the PCB 20. Returning now to FIG. 1, the cable 30 is also affixed to the flexible backing and/or the sheet of graphite and/or the PCB by at least one layer of adhesive. In some embodiments, the at least one layer of adhesive comprises the first layer of adhesive (i.e., the layer that affixes the flexible PCB 20 to the flexible backing 80). In some embodiments, the at least one layer of adhesive comprises the layer of conductive adhesive (i.e., the layer that affixes the sheet of graphite 42 to the PCB 20). And in some embodiments, the at least one layer of adhesive comprises both the first layer of adhesive and the layer of conductive adhesive.

In some embodiments, the cable 30 is affixed to the sheet of graphite 40 and/or the PCB 20 by double-sided foam tape 28 (not shown) that has a front layer of adhesive and a rear layer of adhesive, and at least a portion of the double-sided foam tape 28 is situated between the first region of the PCB 20 and the third region 23 of the PCB.

Although this double-sided foam tape is not depicted in the figures, it would sit between the third region 23 of the PCB 20 and the metal pads 25 on the first region of the PCB 20, both of which are depicted in FIG. 4B.

In the embodiments described above in connection with FIGS. 1-4, and as best seen in FIG. 4A, the second direction D2 (i.e., the direction in which the third region extends from the second region) is substantially perpendicular to the first direction D1 (i.e., the direction in which the second region extends from the first region). But in some alternative embodiments, the second direction D2 is not substantially perpendicular to the first direction D1. One example of this situation is described below in connection with FIGS. 5A-B.

FIGS. 5A and 5B depict another layout for an electrode assembly 200 in which the second direction D2 is substantially parallel to the first direction D1. This sequence of layers in this embodiment is identical to the sequence of layers or the electrode assembly 100 described above in connection with FIGS. 1-4. But the overall shape of the electrode assembly 200 is different (i.e., cloverleaf shaped as opposed to a rounded-V shaped), and the direction in which the third region of the PCB extends from the second region of the PCB is also different. More specifically, FIG. 5A depicts the PCB 20 before it has been folded during the manufacturing process. Notably, the second region 22 extends from the first region of the PCB 20 in a downward direction, and the third region 23 extends from the second region 22 in the same downward direction. FIG. 5B depicts the FIG. 5A embodiment after the second region 22 has been folded (upward) about a horizontal bending axis.

The first region of the PCB 20 includes the at least one metal pad 25 (facing away from the reader in FIG. 5A). The second region 22 is contiguous with the first region and extends from the first region in a first direction D1. And the third region 23 is contiguous with the second region 22 and extends from the second region in a second direction D2 that is parallel to the first direction D1. During the manufacturing process, the PCB 20 is bent back on itself about a bending axis (labeled B) that passes through the second region 22 so that the third region 23 moves toward the reader in FIG. 5A, until the third region 23 reaches the positioned depicted in FIG. 5B. At this point, the third region 23 will be positioned behind the first region of the PCB 20. In some preferred embodiments, the bending radius of the bend that runs through the second region 22 is at least 6 times a thickness of the PCB.

In the example illustrated in FIGS. 5A-B, the bending axis B is substantially perpendicular to the first direction D1, and the second direction D2 is substantially parallel to the first direction. But this is not the only possible configuration. For example, in the FIGS. 5A-B embodiment, the depicted configuration could be replaced with a configuration in which the bending axis B is substantially perpendicular to the first direction D1 and the second direction D2 is substantially perpendicular to the first direction D1. Such a configuration would be similar to the configuration described above in connection with FIGS. 1-4

At a subsequent time in the manufacturing process, the cable 30 is connected (e.g., by soldering) so that it terminates on the third region 23 of the PCB 20, as depicted in FIG. 5B. Note that in the example depicted in FIG. 5B, the cable 30 extends in the second direction D2 from the third region 23 of the PCB 20. Conductive wires within the cable 30 and traces on the PCB 20 form the paths over which the signals from an AC signal generator (not shown) arrive at the metal pads 25 in order to apply TTFields to the region of interest in the subject's body.

In the embodiments described above in connection with FIG. 1-5, the cable 30 is connected (e.g., by soldering) so that it terminates on the third region 23 of the PCB 20. But in some alternative embodiments (not shown) a connector is connected (e.g., by soldering) so that it terminates on the third region 23 of the PCB 20, and this connector mates with a corresponding cable that plugs into the connector. All other features of these alternative embodiments are as described above in connection with FIGS. 1-5.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus comprising:
a flexible backing having a front surface;
a flexible PCB affixed to the front surface of the flexible backing by a first layer of adhesive,
wherein the PCB has (i) a first region having at least one metal pad disposed on one surface of the PCB, (ii) a second region that is contiguous with the first region and extends from the first region in a first direction, and (iii) a third region that is contiguous with the second region and extends from the second region in a second direction,
wherein the PCB is oriented so that the at least one metal pad is front-facing, and
wherein the PCB is bent back on itself about a bending axis that passes through the second region so that the third region is positioned behind the first region;
a sheet of graphite positioned in front of the PCB, wherein the sheet of graphite is affixed to the PCB by a layer of conductive adhesive; and
a cable that terminates on the third region of the PCB,
wherein the cable is affixed to the flexible backing and/or the sheet of graphite and/or the PCB by at least one layer of adhesive.

2. The apparatus of claim 1, wherein the at least one layer of adhesive comprises the first layer of adhesive.

3. The apparatus of claim 1, wherein the at least one layer of adhesive comprises the layer of conductive adhesive.

4. The apparatus of claim 1, wherein the at least one layer of adhesive comprises both the first layer of adhesive and the layer of conductive adhesive.

5. The apparatus of claim 1, wherein the cable is affixed to the sheet of graphite and/or the PCB by double-sided foam tape that has a front layer of adhesive and a rear layer of adhesive, wherein at least a portion of the double-sided foam tape is situated between the first region of the PCB and the third region of the PCB.

6. The apparatus of claim 1, wherein the PCB is bent back on itself with a bending radius that is at least 6 times a thickness of the PCB.

7. The apparatus of claim 1, wherein the bending axis is substantially perpendicular to the first direction.

8. The apparatus of claim 7, wherein the second direction is substantially perpendicular to the first direction.

9. The apparatus of claim 8, wherein the cable extends in the second direction from the third region of the PCB.

10. The apparatus of claim 7, wherein the second direction is substantially parallel to the first direction.

11. An apparatus for applying an electrical signal to a subject's body, the apparatus comprising:
a flexible backing having a front surface;
a flexible PCB affixed to the front surface of the flexible backing by a first layer of adhesive,
wherein the PCB has (i) a first region having at least one metal pad disposed on one surface of the PCB, (ii) a second region that is contiguous with the first region and extends from the first region in a first direction, and (iii) a third region that is contiguous with the second region and extends from the second region in a second direction,
wherein the PCB is oriented so that the at least one metal pad is front-facing, and
wherein the PCB is bent back on itself about a bending axis that passes through the second region so that the third region is positioned behind the first region;
a sheet of graphite positioned in front of the PCB, wherein the sheet of graphite is affixed to the PCB by a layer of conductive adhesive; and
a connector that terminates on the third region of the PCB,
wherein the connector is affixed to the flexible backing and/or the sheet of graphite and/or the PCB by at least one layer of adhesive.

12. The apparatus of claim 11, wherein the at least one layer of adhesive comprises the first layer of adhesive.

13. The apparatus of claim 11, wherein the at least one layer of adhesive comprises the layer of conductive adhesive.

14. The apparatus of claim 11, wherein the at least one layer of adhesive comprises both the first layer of adhesive and the layer of conductive adhesive.

15. The apparatus of claim 11, wherein the connector is affixed to the sheet of graphite and/or the PCB by double-sided foam tape that has a front layer of adhesive and a rear layer of adhesive, wherein at least a portion of the double-sided foam tape is situated between the first region of the PCB and the third region of the PCB.

16. The apparatus of claim 11, wherein the PCB is bent back on itself with a bending radius that is at least 6 times a thickness of the PCB.

17. The apparatus of claim 11, wherein the bending axis is substantially perpendicular to the first direction.

18. The apparatus of claim 17, wherein the second direction is substantially perpendicular to the first direction.

19. The apparatus of claim 18, wherein the connector extends in the second direction from the third region of the PCB.

20. The apparatus of claim 17, wherein the second direction is substantially parallel to the first direction.
